(19) United States
(12) Patent Application Publication
(10) Pub. No.: US 2026/0130735 A1
(43) Pub. Date: May 14, 2026

(54) STETHOSCOPE COVER DISPENSER

(71) Applicant: Stethoscover, LLC, Fort Lauderdale, FL (US)

(72) Inventors: Bradley Fishberger, Fort Lauderdale, FL (US); Jared Franklin, Fort Lauderdale, FL (US); Seth Hochberg, Fort Lauderdale, FL (US)

(21) Appl. No.: 19/386,424

(22) Filed: Nov. 12, 2025

Related U.S. Application Data
(60) Provisional application No. 63/719,748, filed on Nov. 13, 2024.

(51) Int. Cl. A61B 50/00; A61B 7/02

(52) U.S. Cl. CPC A61B 50/00; A61B 7/02; A61B 2050/0058

(57) ABSTRACT

A system for dispensing a cover comprising an adhesive portion for application to a stethoscope is provided. The system comprises a cartridge and a housing. The cartridge comprises a body, a roll of media rotatably coupled to the body, and a first gear operably coupled to the media, such that rotation of the first gear rotates the roll of media. The housing comprises a body, one or more sensors coupled to the housing, and a second gear operably coupled to an actuator disposed within the housing and configured to mesh with the first gear. The one or more sensors are configured to detect a present of an object adjacent the housing. The detection of the presence of the object by the one or more sensors causes dispensing of an adhesive portion of the continuous sheet through a slot of the housing.

### CROSS-REFERENCE TO RELATED APPLICATIONS

This application claims the benefit of U.S. Provisional App. No. 63/719,748, filed on Nov. 13, 2024. The entire contents of which are hereby incorporated by reference.

### BACKGROUND OF THE DISCLOSED SUBJECT MATTER

### Field of the Disclosed Subject Matter

The disclosed subject matter relates to systems and methods for automatically dispensing a cover for a stethoscope to promote sterility thereof; the cover can be manually applied to the stethoscope or directly applied to the stethoscope.

### Description of Related Art

Healthcare professionals routinely use stethoscopes during patient examination. Because the diaphragm or bell of the stethoscope frequently contacts a patient's skin or clothing, it can serve as a vector for cross-contamination between patients if not properly disinfected or covered. Conventional hygiene practices, such as manual wiping or alcohol swabbing, or the use of sleeves applied by hand, can reduce contamination risk but are often time-consuming, inconsistent, and dependent on user diligence.

Accordingly, there remains a need for an automated, contact-free, and replaceable dispensing system that provides consistent presentation of sterile adhesive covers for stethoscopes.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a system for dispensing a cover comprising an adhesive portion for application to a stethoscope, comprising a cartridge comprising a body having an exterior surface and an interior surface; a roll of media rotatably coupled to the body, the roll of media comprising a continuous sheet having removable adhesive portions and non-adhesive portions; and a first gear operably coupled to the roll of media, such that a rotation of the first gear rotates the roll of media; a housing, the housing having a body with an exterior surface and an interior surface, the body configured to receive the cartridge; one or more sensors coupled to the housing and configured to detect a presence of an object adjacent the housing; and a second gear operably coupled to an actuator disposed within the housing, the second gear configured to mesh with the first gear, wherein a rotation of the second gear rotates the first gear, wherein detection of the presence of the object by the one or more sensors causes dispensing of an adhesive portion of the continuous sheet through a slot of the housing.

In some embodiments, the housing further comprises a lid pivotably coupled to the body of the housing.

In some embodiments, the lid comprises a curved surface.

In some embodiments, the slot is disposed tangentially relative to a curvature of the lid, such that an adhesive portion of the continuous sheet is dispensed along the curved surface of the lid.

In some embodiments, the one or more sensors are disposed on the lid.

In some embodiments, the system further comprises a protrusion disposed adjacent the slot, the one or more sensors disposed between the slot and the protrusion.

In some embodiments, the system further comprises a third gear operably coupled between the actuator and the second gear.

In some embodiments, the actuator is directly coupled to the third gear such that rotation of an output shaft of the actuator directly rotates the third gear.

In some embodiments, the third gear is configured to mesh with the second gear.

In some embodiments, the adhesive portion comprises a circular region and a linear region extending from the circular region.

In some embodiments, the system further comprises a machine-readable code, the machine-readable code disposed on the exterior surface of the housing.

In some embodiments, the cartridge further comprises a plurality of mounting projections extending outwardly from the exterior surface of the body of the cartridge.

In some embodiments, the housing further comprises a plurality of guide slots formed on the interior surface of the body of the housing, each one of the plurality of guide slots configured to receive one of the plurality of mounting projections of the cartridge.

In some embodiments, the system further comprises one or more sensors coupled to the cartridge, the one or more sensors configured to detect a predetermined opacity of the continuous sheet.

In some embodiments, the system further comprises a first electrical interface disposed within the housing and a second electrical interface disposed on the cartridge, the first electrical interface and the second electrical interface configured to engage and electrically communicate when the cartridge is received within the housing.

The disclosed subject matter also includes a cartridge for use in a system for dispensing a cover comprising an adhesive portion for application to a stethoscope, comprising a body having an exterior surface and an interior surface; a roll of media rotatably coupled to the body, the roll of media comprising a continuous sheet having adhesive portions and non-adhesive portions; and a first gear operably coupled to the roll of media, such that a rotation of the first gear rotates the roll of media.

In some embodiments, the body comprises a pair of opposing side panels and a plurality of cross-members extending between the side panels.

In some embodiments, the cartridge further comprises a take-up spindle disposed within the body and operably coupled to the first gear.

In some embodiments, the cartridge further comprises a spindle shell disposed over the take-up spindle.

In some embodiments, a first end of the continuous sheet is coupled (e.g., removably coupled) to the spindle shell.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1A illustrates a front perspective view of a housing of a system for dispensing a cover on a stethoscope, in accordance with embodiments of this disclosure.

FIGS. 1B and 1C illustrate side views of a housing as shown in FIG. 1A, in accordance with embodiments of this disclosure.

FIG. 1D illustrates a rear view of a housing as shown in FIG. 1A, in accordance with embodiments of this disclosure.

FIG. 1E illustrates a front view of a housing as shown in FIG. 1A, in accordance with embodiments of this disclosure.

FIG. 1F illustrates a top view of a housing as shown in FIG. 1A, in accordance with embodiments of this disclosure.

FIG. 1G illustrates a bottom view of a housing as shown in FIG. 1A, in accordance with embodiments of this disclosure.

FIG. 2 illustrates an exploded assembly view of a system for dispensing a cover on a stethoscope, in accordance with embodiments of this disclosure.

FIGS. 3A-3B illustrate perspective views of a cartridge of a system for dispensing a cover on a stethoscope, in accordance with embodiments of this disclosure.

FIGS. 3C-3D illustrate side views of a cartridge as shown in FIGS. 3A-3B , in accordance with embodiments of this disclosure.

FIG. 3E illustrates a top view of a cartridge as shown in FIGS. 3A-3B , in accordance with embodiments of this disclosure.

FIG. 3F illustrates a bottom view of a cartridge as shown in FIGS. 3A-3B , in accordance with embodiments of this disclosure.

FIG. 3G illustrates a front view of a cartridge as shown in FIGS. 3A-3B , in accordance with embodiments of this disclosure.

FIG. 3H illustrates a rear view of a cartridge as shown in FIGS. 3A-3B , in accordance with embodiments of this disclosure.

FIGS. 4A-4B illustrate interior side views of panels of a housing as shown in FIG. 1A, in accordance with embodiments of this disclosure.

FIGS. 5A-5B illustrate interior side views of a housing as shown in FIG. 1A, with one housing panel omitted for clarity to show interior components, in accordance with embodiments of this disclosure.

FIGS. 6A-6B illustrate interior side views of a system, including a housing, as shown in FIG. 1A, and a cartridge, as shown in FIGS. 3A-3B , with one housing panel omitted for clarity to show interior components, in accordance with embodiments of this disclosure.

FIG. 7 illustrates a schematic top view of a portion of a continuous sheet of a roll of media, in accordance with embodiments of this disclosure.

FIG. 8 illustrates a schematic side view of a path of a continuous sheet of a roll of media within the cartridge, in accordance with embodiments of this disclosure.

### DETAILED DESCRIPTION

The various concepts introduced above and discussed in greater detail below may be implemented in a number of ways, as the described concepts are not limited to any particular manner of embodiment.

Reference will now be made in detail to the exemplary embodiments of the present disclosure, examples of which are illustrated in the accompanying drawings. Wherever possible, the same reference numbers will be used throughout the drawings to refer to the same or like parts.

References herein to positions of elements (e.g., “top”, “bottom”) are merely used to describe the orientation of various elements in the Figures. It should be noted that the orientation of various elements may differ according to other embodiments, and that such variations are intended to be encompassed by the present disclosure.

The term “about” means a range of values inclusive of the specified value that a person of ordinary skill in the art would reasonably consider to be comparable to the specified value. In some embodiments, “about” means within a standard deviation using measurements generally accepted by a person of ordinary skill in the art. In some embodiments, “about” means ranging up to ±10% of the specified value. In some embodiments, “about” means ranging up to ±5% of the specified value. In some embodiments, “about” means the specified value.

The disclosed subject matter relates to systems and methods for automatically dispensing a cover (e.g., a sterile cover) on a stethoscope. The system can include a housing configured to receive a removable and replaceable cartridge containing a roll of media. The housing can include one or more sensors configured to detect a user or a stethoscope and to interact with the cartridge to control dispensing of adhesive portions of the roll of media. An actuator within the housing can drive a gear train that rotates the roll of media to advance a continuous sheet having non-adhesive portions and adhesive portions. The adhesive portion (e.g., cover) can include an adhesive surface and an opposing non-adhesive surface, such that the adhesive surface adheres to the stethoscope diaphragm while the opposing, non-adhesive surface remains exposed to contact a patient or ambient environment. An adhesive portion can be dispensed through a slot in the housing so that a user may press the stethoscope diaphragm against the exposed portion and remove the adhesive portion, thereby applying the cover to the stethoscope diaphragm.

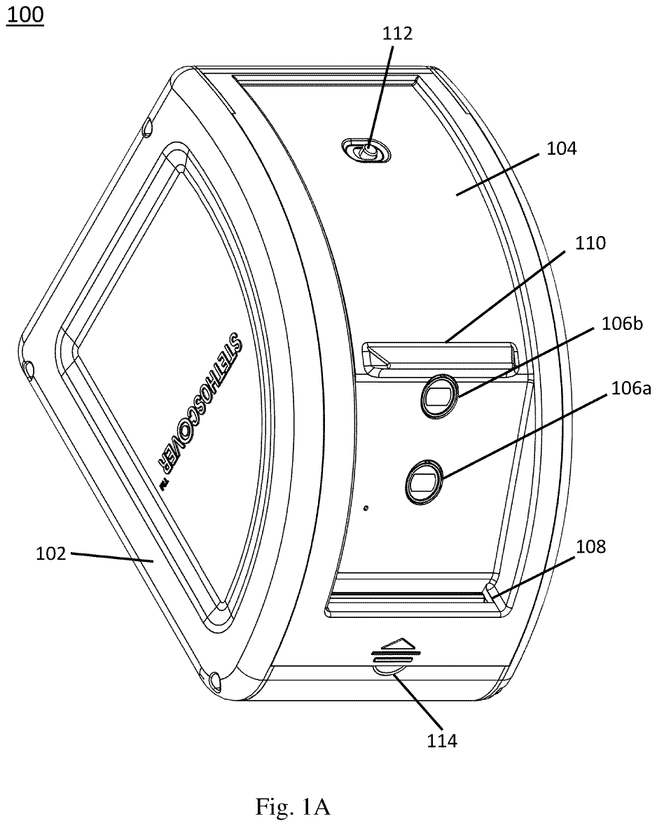

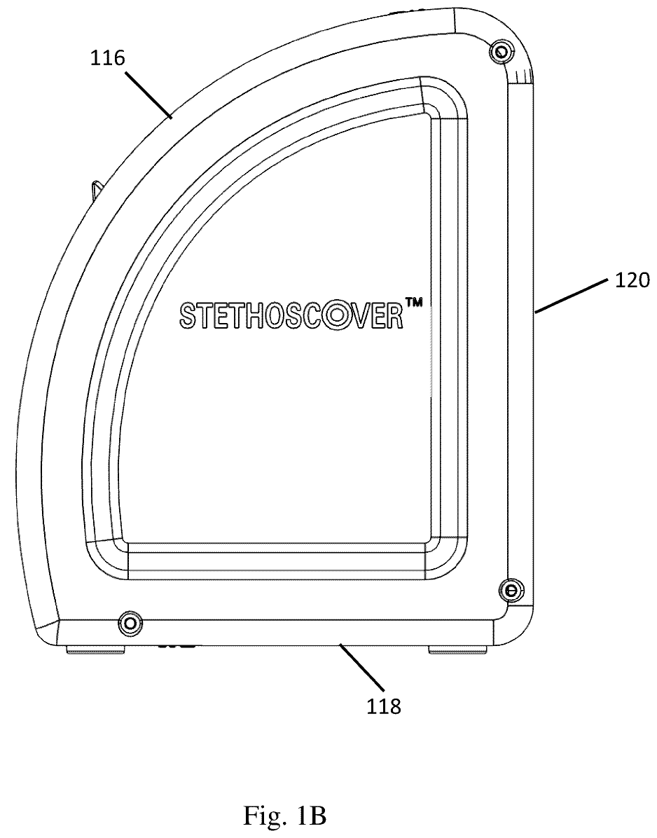

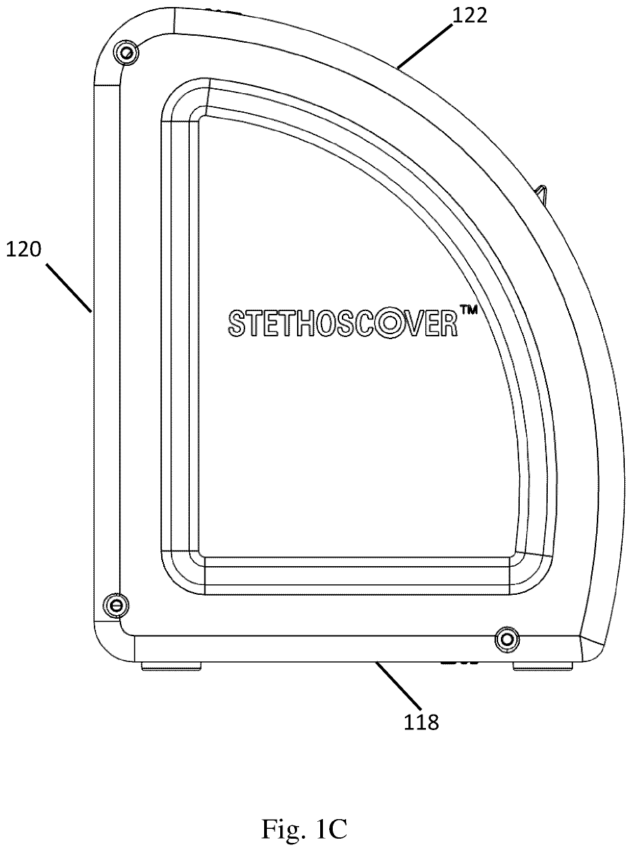

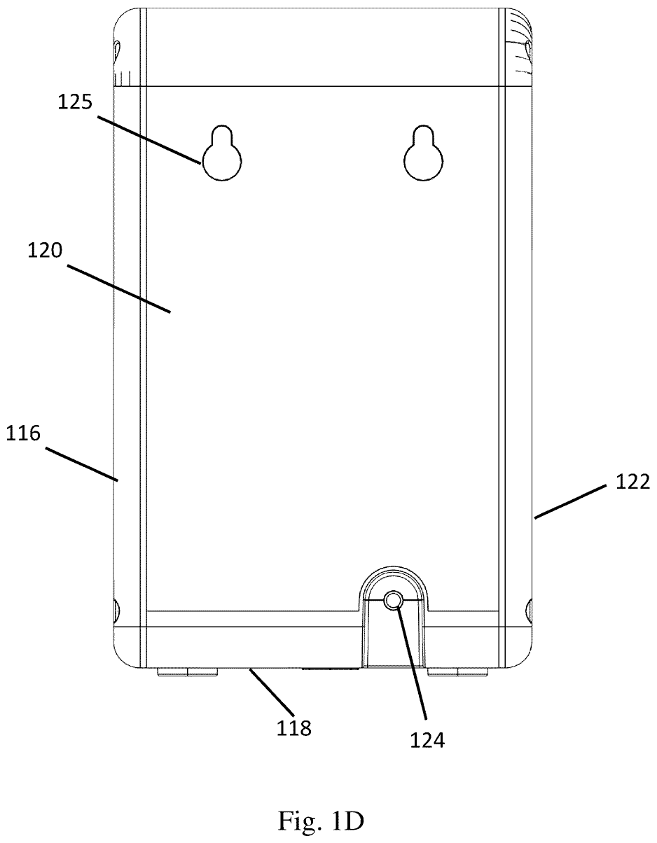

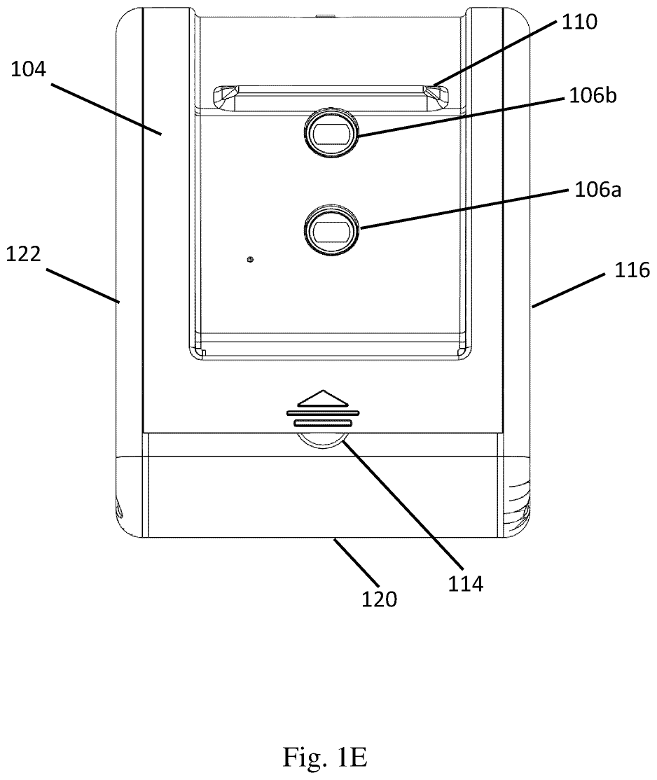

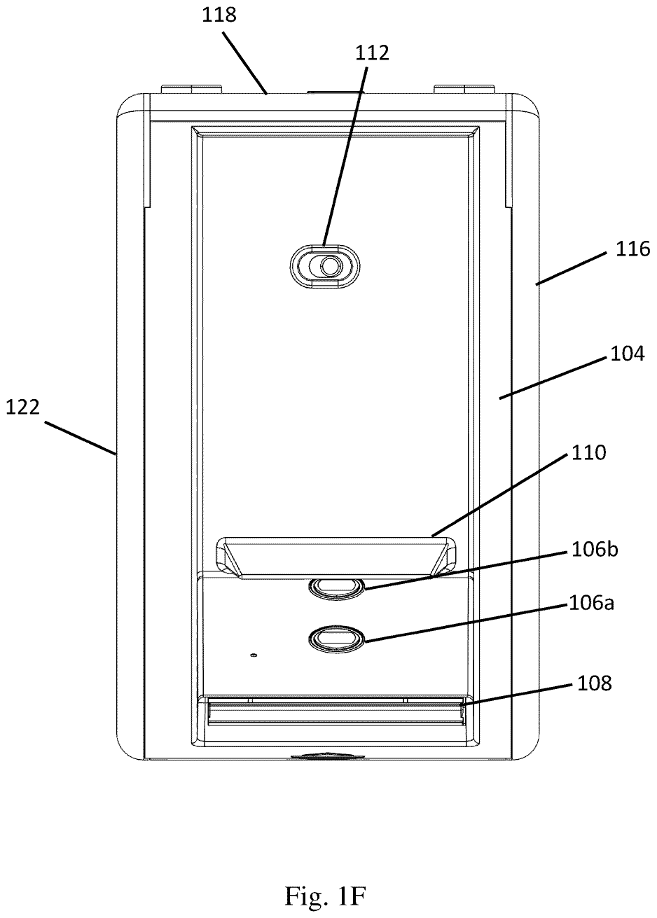

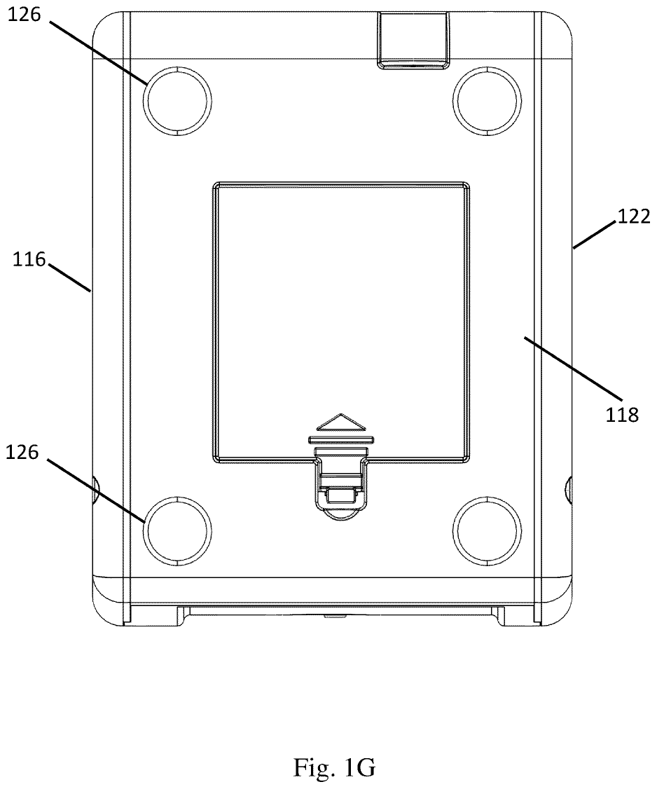

Referring now to FIGS. 1A-1G , various exterior views of a housing 100 for dispensing a stethoscope cover are illustrated. As shown in FIG. 1A, the system includes a housing 100 having a body 102 defining an interior cavity configured to receive a cartridge. The body 102 provides a structural enclosure for the system and supports internal components.

The housing 100 can include a lid 104 pivotably coupled (e.g., hingedly) to the body 102. The lid 104 can hinge along an edge of the housing to provide access for inserting or removing the cartridge 300. In some embodiments, the lid 104 includes detents to secure the lid 104 to the body 102 in a locked or closed configuration (e.g., as shown in FIG. 1A). In some embodiments, the body 102 is rectangular with rounded or radiused corners. In some embodiments, the body 102 has a quarter-circular shape when view from the side, with one arcuate section and adjacent planar sides. The lid 104 can match the shape of the body 102 and can be curved or planar.

The body 102 can be formed of multiple side panels (e.g., two or more side panels). For example, the housing 100 can include a first panel 116, a second side panel 118, a third panel 120, and a fourth side panel 122. The first and fourth side panels 116, 122 can define the lateral sides of the housing; the second side panel 118 can define the base of the housing; and the third side panel 120 can define the rear of the housing. In some embodiments, the side panels of the housing (e.g., side panels 116, 118, 120, 122) are integrally formed as a single-piece body. In some embodiments, the side panels of the housing are discrete components assembled together to define the housing body 102. The side panels can be formed from a rigid material (e.g., metal, plastic). The side panels can include reinforcing ribs or bosses.

One or more sensors (e.g., sensors 106a, 106b) can be mounted on the housing 100. In some embodiments, the one or more sensors are mounted on (e.g., coupled to) the body 102 when the slot 108 and protrusion 110 are formed on the body 102. In some embodiments, the one or more sensors are mounted on the lid 104 when the slot 108 and protrusion 110 are formed on the lid 104. The one or more sensors can be disposed between the slot 108 and protrusion 110. For example, the lid 104 can include a first sensor 106a and a second sensor 106b spaced apart from another along the surface of the lid 104. The first sensor 106a and second sensor 106b can be aligned along a path extending from the slot 108 to the protrusion 110.

In some embodiments, the one or more sensors (e.g., sensors 106a, 106b) are optical sensors, infrared sensors, or capacitive proximity sensors configured to detect the presence of an object, such as a user's hand or a stethoscope. In some embodiments, the one or more sensors are configured to detect the presence of an object positioned about 2 inches from the one or more sensors. In some embodiments, the sensors are calibrated to distinguish between a user's hand motion, a stethoscope, and environmental interference.

Providing two sensors can improve object detection reliability and accuracy. For example, in some embodiments, one sensor can detect an approaching object while the other sensor can confirm its distance or alignment relative to the housing. In another example, in some embodiments, the two sensors operate in a redundant mode, such that both sensors perform the same detection function. For example, each sensor can emit and detect light within a defined field of view to determine whether an object is present. The sensors can be configured such that both must detect an object (e.g., each sensor senses a reduction in reflected light intensity or an interruption of emitted light) before the control system initiates a dispensing operation. This redundant mode configuration can prevent activation if only one sensor is partially obstructed or if transient reflections occur, thereby increasing reliability and ensuring that dispensing occurs only when a user's hand or stethoscope diaphragm is positioned in the intended area. In various aspects, the two sensors can operate cooperatively to validate that a detected object corresponds to an event requiring dispensing of a cover, thereby preventing false triggering. Additionally, in some embodiments each sensor is configured with the same field of view (e.g. range of sensitivity); in some embodiments the sensors can be configured with different fields of view.

The housing 100 can include a slot 108 (e.g., rectangular slot) defining an outlet through which an adhesive portion (e.g., cover) of a roll of media is dispensed (while keeping the remaining media within the housing). The protrusion 110 can be positioned adjacent the slot 108, for example, along a curved surface of the lid 104. In some embodiments, the protrusion 110 serves as a handle that can be grasped to move the lid. In some embodiments, the protrusion 110 serves as a stopper or support feature that retains the dispensed adhesive portion between the slot 108 and the protrusion 110. For example, when the housing 100 is positioned on tabletop or other horizontal surface, and the adhesive portion is dispensed along the curved contour of the lid 104, gravity may cause the adhesive portion to slide downward towards the horizontal surface. The protrusion 110 can prevent such sliding and provides a support surface for the adhesive portion until it is pressed against by the stethoscope diaphragm and removed. Thus the cover can be maintained in a fixed and predictable location, so that a user can quickly and accurately apply the cover to the stethoscope by engaging the stethoscope with the known target area of the device. In some embodiments, the slot 108 is formed on the lid 104. In some embodiments, the slot 108 is formed on the body 102. In some embodiments, the protrusion 110 is disposed on the lid 104. In some embodiments, the protrusion 110 is disposed on the body 102. In some embodiments, the protrusion 110 is integrally formed as part of the lid curvature. In some embodiments, the protrusion 110 is provided as a removable or replaceable component.

The slot 108 can be oriented such that the adhesive portion of the media is dispensed along a surface of the lid 104 (rather than through the thickness of the lid). For example, the slot 108 can be orientated such its length extends transversely across the lid 104, and its opening is angled relative to the thickness of the lid. In some embodiments, the lid 104 includes a recessed portion or dispensing channel that extends between the slot 108 and the protrusion 110. The slot 108 can open into this recessed portion so that, during dispensing, the adhesive portion travels along the surface (e.g., curved surface) of the lid 104. In this way, the adhesive portion follows the contour of the lid 104 and remains supported on its exterior (non-adhesive surface) as it exits the housing. In various aspects, this arrangement prevents the dispensed portion from hanging freely or folding under gravity (avoiding undesired displacement or damage to the cover) and allows the protrusion 110 to support or stop the adhesive portion at a defined position for application to the stethoscope.

In some embodiments, the housing 100 includes a power control element 112 (e.g., switch, button) positioned on the lid 104. In some embodiments, the power control element 112 is positioned on a side panel of the housing. The power control element 112 can be mechanically or electronically coupled to a control system within the housing and cab be configured to toggle power to the system or activate a standby or calibration mode. In some embodiments, the power control element 112 includes a tactile or illuminated indicator that provides visual or haptic feedback when the system is powered, when a dispensing operation (dispensing of a cover) is in progress, or when the dispensing operation is complete.

In some embodiments, the second side panel 118 (e.g., bottom of the housing) includes a notch 114. In some embodiments, the lid 104 includes a notch 114. The notch 114 can define a cutout or recessed portion of either the lid 104 or the second side panel 118 such that, when the lid 104 is in a closed configuration, the cutout forms a gap or space between the lid and the housing body 102. The gap can allow user to insert a finger to grasp and lift the lid 104 to an open position.

In some embodiments, the housing 100 includes a power-adapter receiver 124 positioned on a side panel of the body 102 (e.g., side panel 116, 118, 120, 122). The power-adapter receiver 124 can be configured to receive a plug or connector from an external power source to supply electrical power to the system (e.g., the one or more sensors, actuator, etc.). In some embodiments, the power-adapter receiver 124 is recessed within the housing to protect the connector interface. In some embodiments, the system can operate using one or more internal batteries.

In some embodiments, the housing 100 includes one or more features 125 (e.g., openings, recesses) configured to facilitate mounting or securing of the housing to an external structure. For example, the housing 100 can include one or more features 125 formed in the third side panel 120. The one or more features 125 can receive fasteners or engage complementary projections to allow the housing 100 to be affixed to a wall, support frame, or stand. In some embodiments, the one or more features 125 are through-holes sized to receive screws or bolts. In some embodiments, the one or more features 125 are shaped as keyhole or slot-type mounting features.

In some embodiments, the housing 100 includes one or more supporting protrusions 126 extending from the side panel 118. The supporting protrusions 126 can define feet or stand-off elements that stabilize the housing when placed on a tabletop or other support surface. In some embodiments, the supporting protrusions 126 maintain a clearance between the housing 100 and the underlying surface. In some embodiments, the supporting protrusions 126 engage corresponding recesses to secure the housing 100 in a fixed position, such as on a wall or equipment cart.

In some embodiments, the housing 100 includes a machine-readable code, such as a QR code, printed, engrave, or affixed to an exterior surface of the housing body 102 or lid 104. The QR code can serve as an identification, tracking, or communication feature for maintenance and identification purposes. For example, the QR code can encode information identifying the model number, serial number, or production lot of the system, and can link to a remote database or cloud-based service that maintains cartridge usage logs, cleaning schedules, or performance history. Scanning the QR code with a compatible device (e.g., smart phone, tablet, etc.) can prompt the display of relevant maintenance instructions on a screen of the compatible device or prompt ordering of cartridges and/or rolls of media.

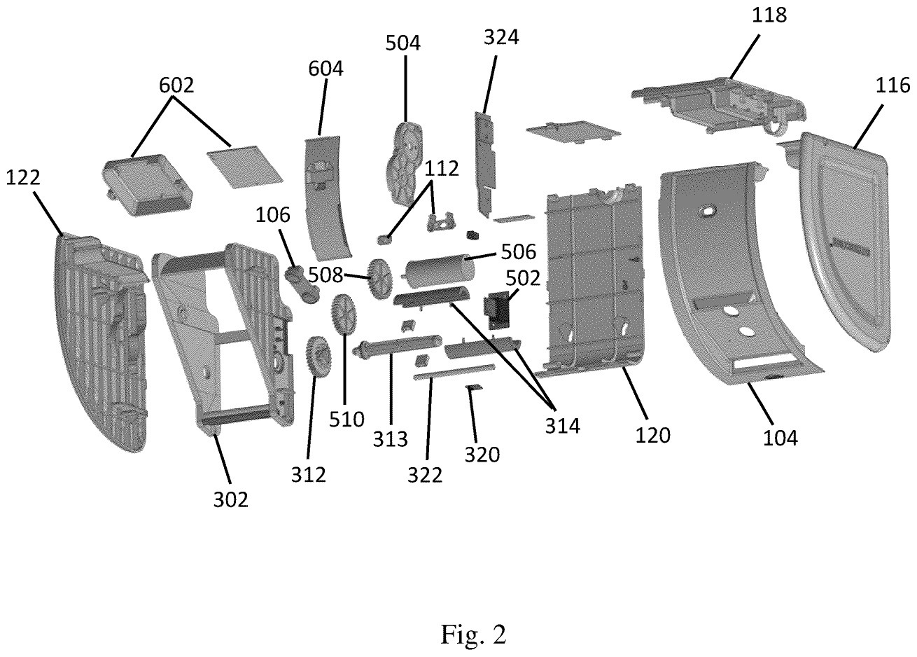

Referring now to FIG. 2, an exploded view of a system for dispensing a stethoscope cover is illustrated. FIG. 2 illustrates components of the housing 100 and the removable, replaceable cartridge.

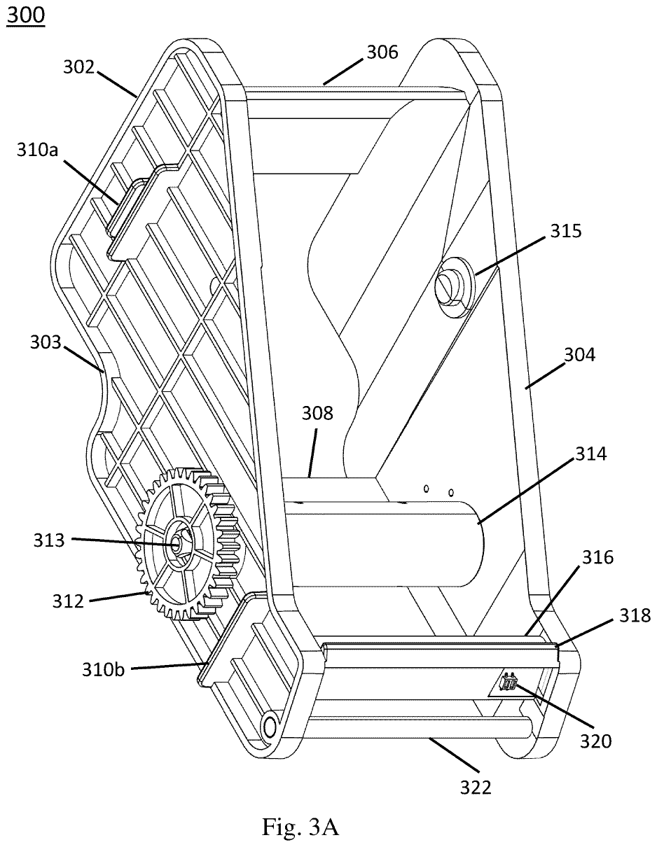

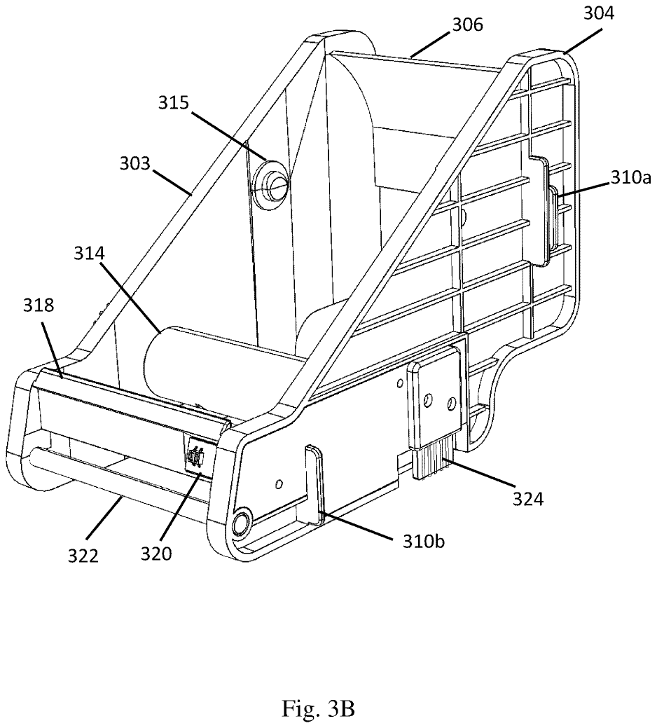

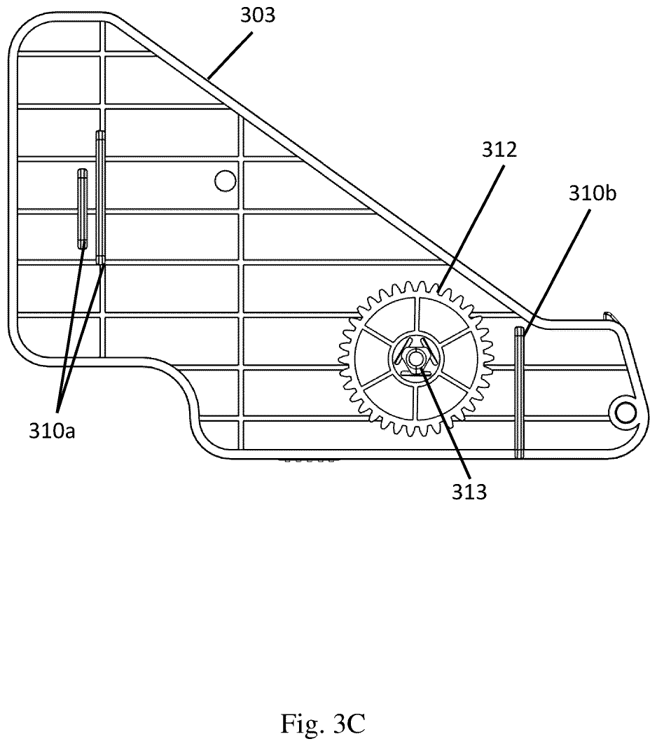

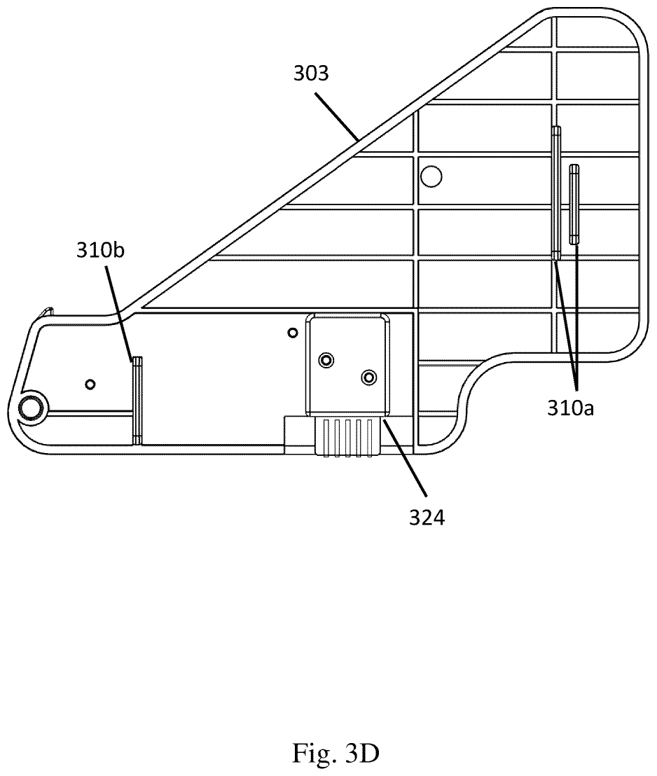

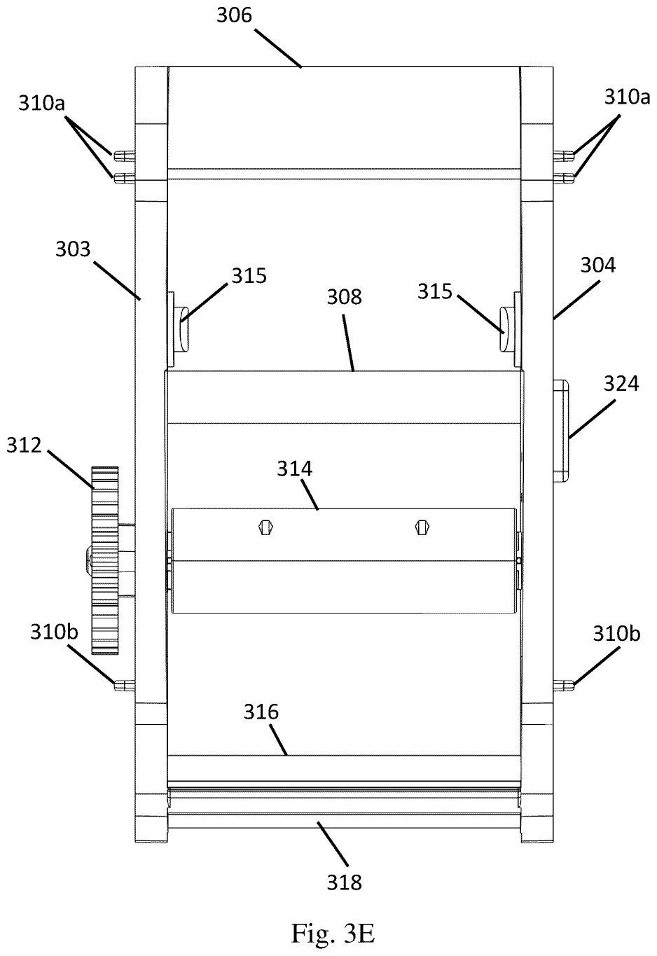

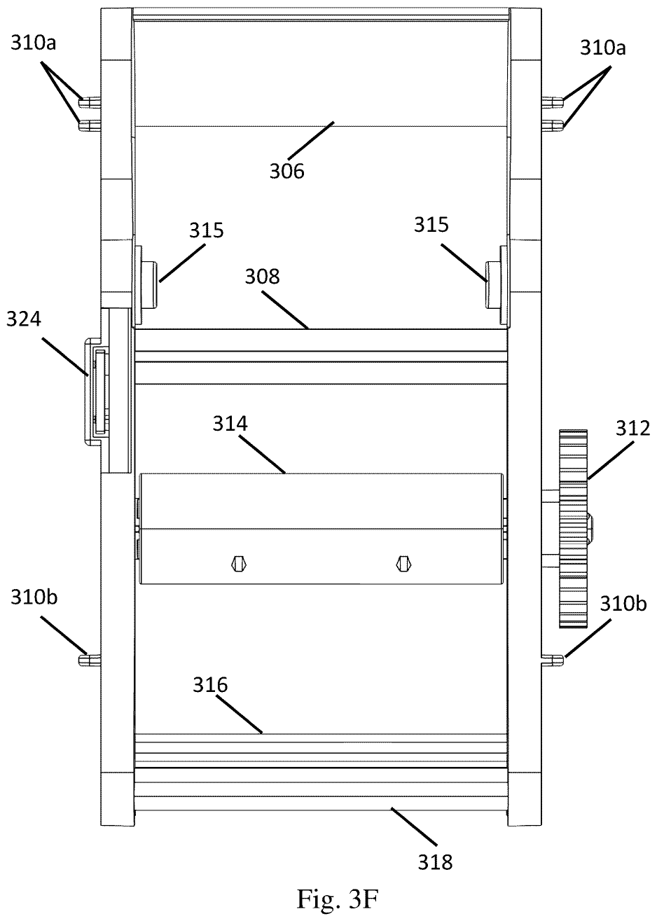

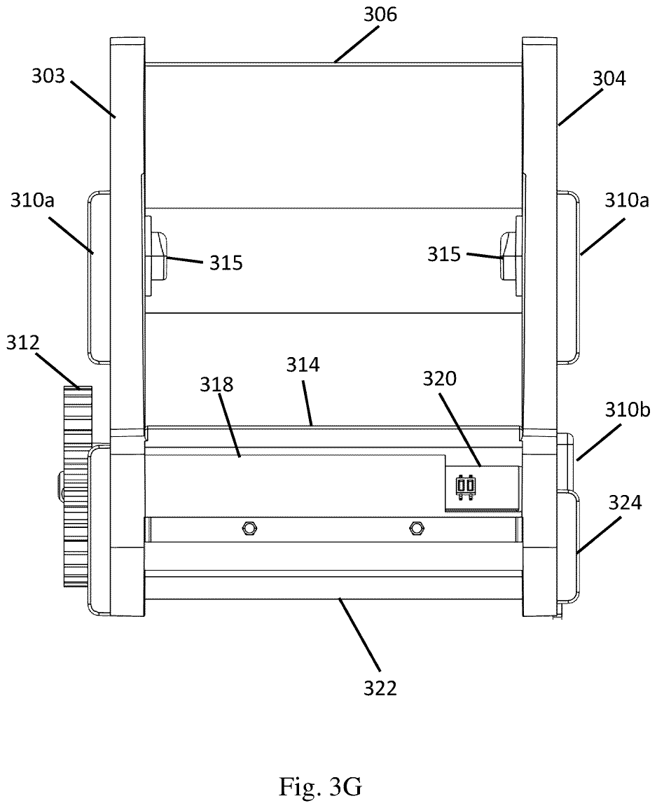

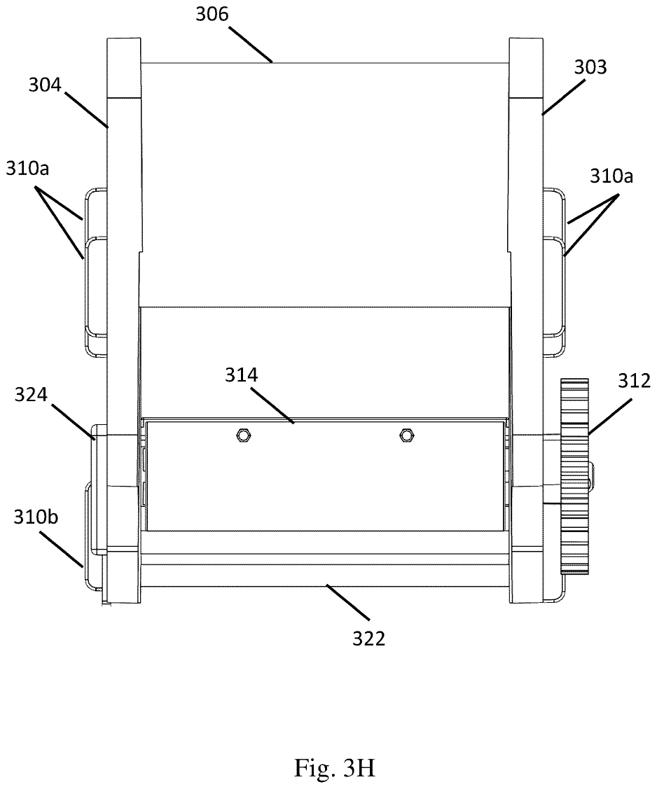

Referring now to FIGS. 3A-3H , various views of a cartridge 300 configured for use within the housing 100 are illustrated. As shown in FIGS. 3A-3H , the cartridge 300 includes a body 302 defining an interior region for receiving and supporting a roll of media. The cartridge body 302 can include a first side panel 303 and a second side panel 304 connected by a first cross-member 306 and a second cross-member 308, which together provide a rigid frame. In some embodiments, the side panels and the cross-members of the cartridge body 302 are integral. In some embodiments, the side panels and the cross-members of the cartridge body 302 are discrete components joined by fasteners or interlocking features (e.g., snap-fittings).

The cartridge 300 can include one or more mounting projections (e.g., rectangular projections) extending outward from exterior surfaces of the cartridge body 302. For example, the one or more mounting projections can include a first set of mounting projections 310a and a second set of mounting projections 310b configured to be received within corresponding guide slots 402a, 402b of the housing 100 to guide cartridge during insertion and to secure its position during operation. In some embodiments, the first set of mounting projections 310a includes at least one projection (e.g., two) on each side panel 303, 304 that extends outward from the cartridge body 302. In some embodiments, the second set of mounting projections 310b includes at least one projection (e.g., two) on each side panel 303, 304 that extends outward from the cartridge body 302. In some embodiments, the first set of mounting projections 310a are positioned toward an upper region of the cartridge body, while the second set of mounting projections 310b are spaced apart from the first set (e.g., positioned toward a lower region of the cartridge body 302). The one or more projections can each be rectangular, cylindrical, or key to match the geometry of the corresponding guide slots and ensure repeatable positioning of the cartridge 300 within the housing 100.

A cartridge gear 312 can be operably to a removable and replaceable roll of media installed within the cartridge body 302 through a take-up spindle 313. The take-up spindle 313 can define a central shaft that extends between the side panels 303, 304, and can include an outer surface for receiving the non-adhesive portions of the roll of media. In some embodiments, the take-up spindle 313 includes at least one spindle shell 314, such as a cylindrical sleeve or two cylindrical half shells, fixedly coupled to the spindle and enclosing a portion of the spindle. The two cylindrical half-shells can be attached to one another around the spindle 313 so as to enclose the spindle and define an outer winding surface. The at least one spindle shell 314 can coupled to the spindle 313 or to another spindle shell 314 by a press-fit, snap-fit, or keyed engagement. The outer surface of the spindle 313, or of the spindle shell 314, can be textured, ribbed, or contoured to promote winding of the continuous sheet and prevent slippage as the diameter of the take-up roll increases during dispensing.

The cartridge 300 can include roll-supporting features 315 configured to rotatably support opposite ends of a shaft (e.g., solid shaft, hollow shaft) on which the roll of media is disposed. For example, in some embodiments, the roll-supporting features 315 include a pair of boss features (e.g., cylindrical boss features) that extend inwardly from interior surfaces of the side panels 303, 304 of the cartridge body 302, each boss feature being configured to receive a respective end of the shaft. In some embodiments, the roll-supporting features 315 include a pair of recesses (e.g., cylindrical recesses) that extend at least partially through the thickness of the side panels 303, 304, each recess being configured to receive a respective end of the shaft. In some embodiments, the roll-supporting features 315 include a recess disposed on one side panel of the cartridge body 302 and a boss feature disposed on an opposing side panel, such that together the recess and boss feature rotatably support the opposite ends of the shaft.

A roll of media can be rotatably supported within the cartridge 300 by the roll-supporting features 315. The roll of media can include a continuous sheet having both adhesive portions and non-adhesive portions. The continuous sheet can function as a liner or carrier substrate, with the adhesive portions removably attached to a release-coated surface of the liner at spaced intervals along its length. Each adhesive portion can define an individual cover configured to adhere to a stethoscope or other surface, while the non-adhesive portions of the same sheet form continuous regions of the liner that support the adhesive portions and separate adjacent adhesive portions along the continuous sheet. In some embodiments, the liner can be formed from a polymeric or paper substrate having a low-energy release surface to allow the adhesive portions to detach cleanly when dispensed, and an opposite surface that provides structural stiffness and traction for advancing the sheet within the cartridge.

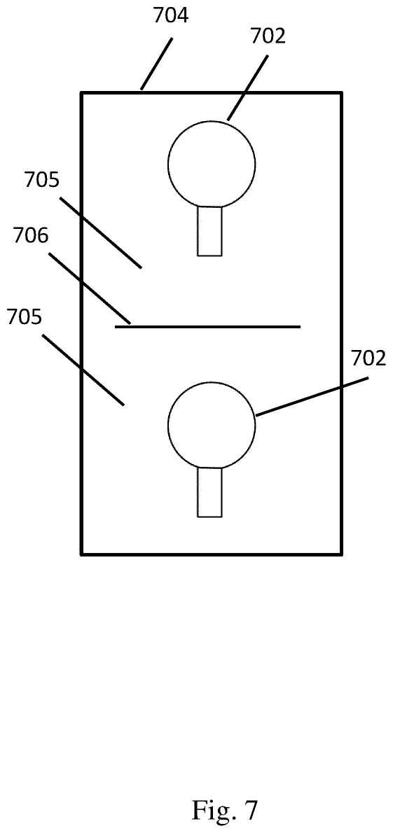

The cover can have a circular portion and an integral linear extension that projects from the circular portion (e.g., as shown in FIG. 7). The linear extension can serve as a tab to facilitate handling, peeling, or alignment of the cover during dispensing and application.

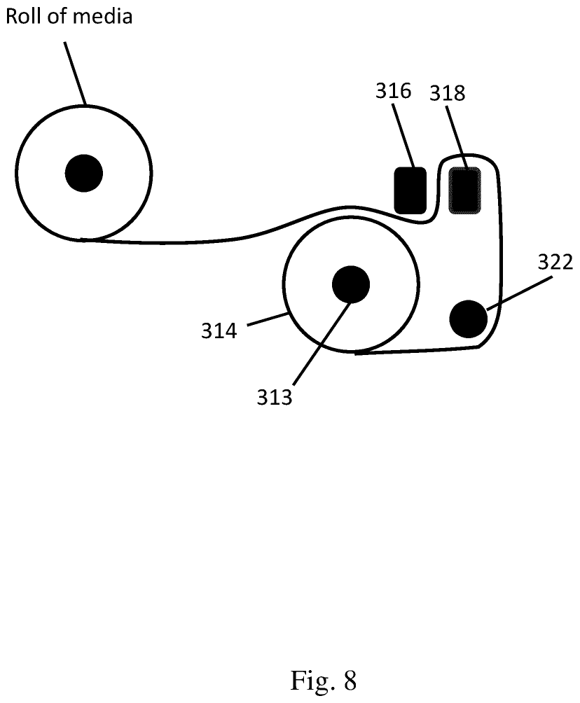

The cartridge 300 can include cross-members that guide and support the continuous sheet within the cartridge body 302. As shown, for example, in FIG. 3A, with added reference to FIG. 8, a third cross member 316 (e.g., rectangular bar member) and a fourth cross-member 318 can extend between the side panels 303, 304 and define a gap or passage through which the continuous sheet is directed. The sheet can pass from the roll of media supported on the roll-supporting features 315 and through the gap defined between the third and fourth cross-members 316, 318. As the sheet moves through this region, an adhesive portion of the sheet can detach from the sheet, allowing the adhesive portion to be dispensed separately while the sheet continues along the media path (i.e., path that the continuous sheet follows). The sheet can then pass over the fourth cross-member 318, such that it covers the one or more sensors 320 disposed thereon, and subsequently pass over a rod 322 positioned adjacent (e.g., below) the fourth cross-member 318. The sheet can be directed rearward toward the take-up spindle 313, where the continuous sheet is wound over the outer surface (e.g., outer surface of the take-up spindle 313 or outer surface of the at least one spindle shell 314 disposed over the take-up spindle). In various aspects, this arrangement can maintain a controlled media path, ensuring that the sheet is properly tensioned and aligned as it travels through the cartridge. A first end of the continuous sheet can be fed through the media path (e.g., over the take-up spindle 313, through the gap between the cross-members 316, 318, over the cross-member 318 and rod 322 (e.g., metal rod), and directed rearward toward the take-up spindle 313) and secured to the take-up spindle 313 or spindle shell 314, for example, by insertion into a slot, clipping engagement, or frictional fit, so that rotation of the spindle winds the sheet as dispensing occurs.

The fourth cross-member 318 can include one or more sensors 320 configured to detect characteristics of the continuous sheet and to control dispensing of the adhesive portions. The one or more sensors 320 can be positioned to face the sheet as it passes over the cross-member 318. In some embodiments, the sensors 320 are optical, infrared, capacitive, reflective, or transmissive sensors, or combinations thereof, configured to detect indicia on the sheet, such as a change in color, contrast, reflectivity, opacity, or a printed marking (e.g., a line or symbol disposed between adjacent adhesive portions). Detection of such indicia can be used to determine a boundary between successive adhesive portions and to stop rotation of the take-up spindle 313 once a single adhesive portion has been dispensed. In some embodiments, the one or more sensors 320 detects an absence or depletion of the sheet to indicate that the roll of media has been exhausted. The one or more sensors 320 can communicate with a control system disposed within the housing 100 through an electrical interface 324 (e.g., chip reader) of the cartridge, allowing the control system to dynamically adjust actuator operation or halt dispensing in response to sensor feedback. The sensor 320 can also detect debris or contamination that may be present on a cover, and automatically advance the sheet so the next cover is advanced to the target zone on the for application to the stethoscope, thereby avoiding use of a contaminated cover and ensuring a hygienic stethoscope for subsequent use.

The electrical interface 324 can be configured to establish electrical communication between the cartridge 300 and the housing 100 when the cartridge is installed. The electrical interface 324 can be disposed along an exterior surface of the cartridge body 302 (e.g., exterior of side panel 304) and can be mechanically coupled to the cartridge via a bracket or mounting support. The bracket can position the electrical interface 324 such that, upon insertion of the cartridge 300 into the housing 100, the interface 324 aligns and mates with a corresponding electrical interface 502 of the housing to form an electrical connection. In some embodiments, the electrical interface 324 can include contact pads, spring-biased pins, conductive terminals, or plug-type connectors configured to transmit electrical signals between the cartridge 300 and the housing 100. The electrical interface 324 can provide communication between the one or more sensors (e.g., sensors 106a, 106b, 120) and the control system, thereby enabling sensor feedback to control actuation and dispensing operations. In some embodiments, the electrical interface 324 can also facilitate power transfer from the housing to electrical components within the cartridge (e.g., one or more sensors 320).

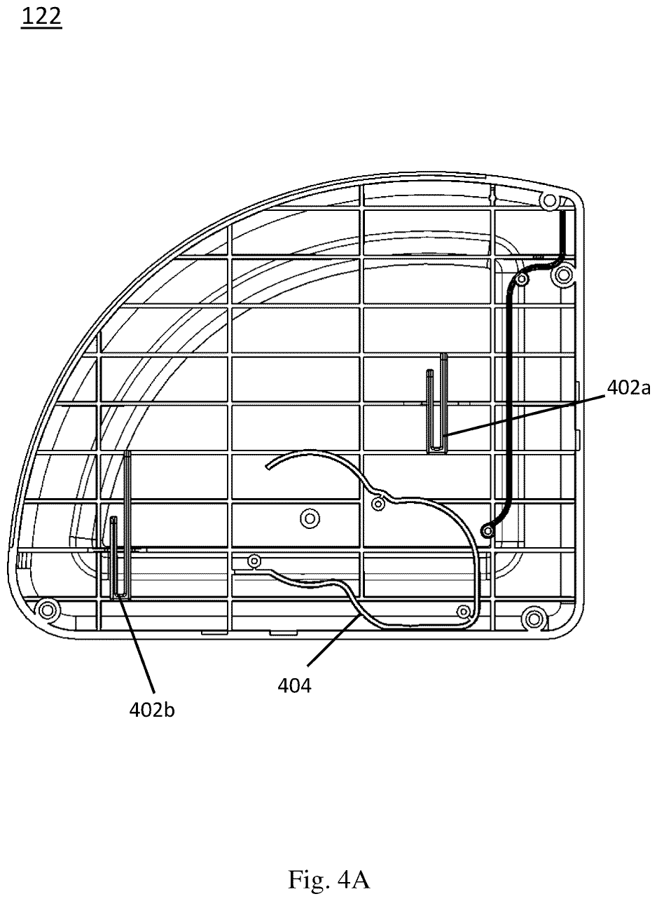

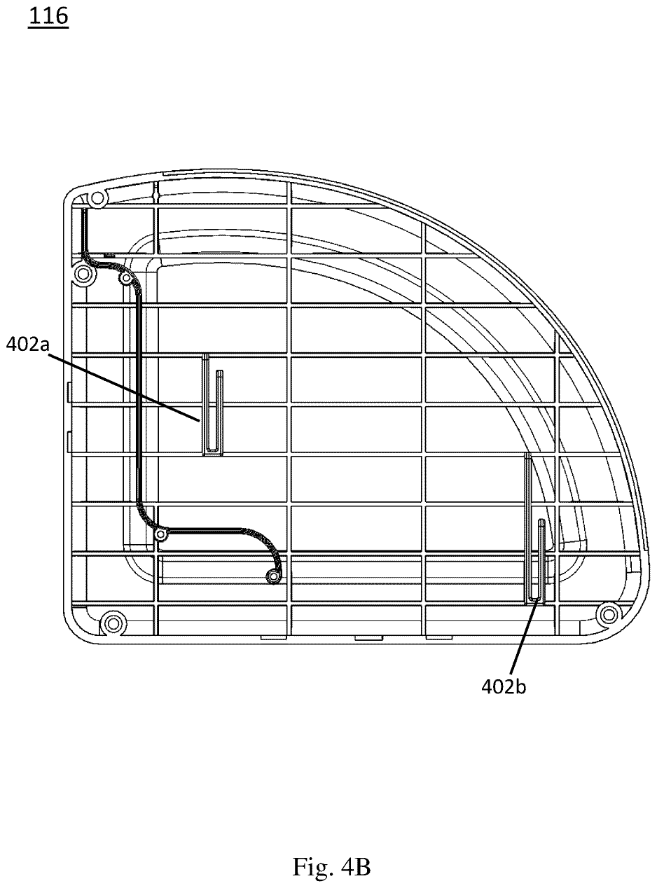

Referring now to FIGS. 4A-4B , interior side views of the side panels 116, 122 of the housing 100, as shown in FIG. 1A, are shown. As shown, the first and fourth side panel 116, 122 can define an interior surface configured to support or align with components of the cartridge 300 and the internal drive assembly (e.g., including gear train and actuator). The interior surfaces can include one or more guide slots 402a, 402b configured to receive the corresponding mounting projections 310a, 310b of the cartridge 300. In some embodiments, the one or more guide slots are U-shaped extending inwards from the inner surface of the panel toward the interior of the housing 100. The one or more guide slots 402a, 402b of one side panel can be laterally aligned with the corresponding guide slots 402a, 402b of the opposing side panel such that the mounting projections 310a, 310b are simultaneously received within the aligned pair of guide slots on opposite side of the housing body 102. The one or more guide slots 402a, 402b can extend partially along the height of the panels and can facilitate sliding insertion and guided positioning of the housing body 102. The fourth side panel 122 can include a frame region 404 configured to receive and support the actuator and gears.

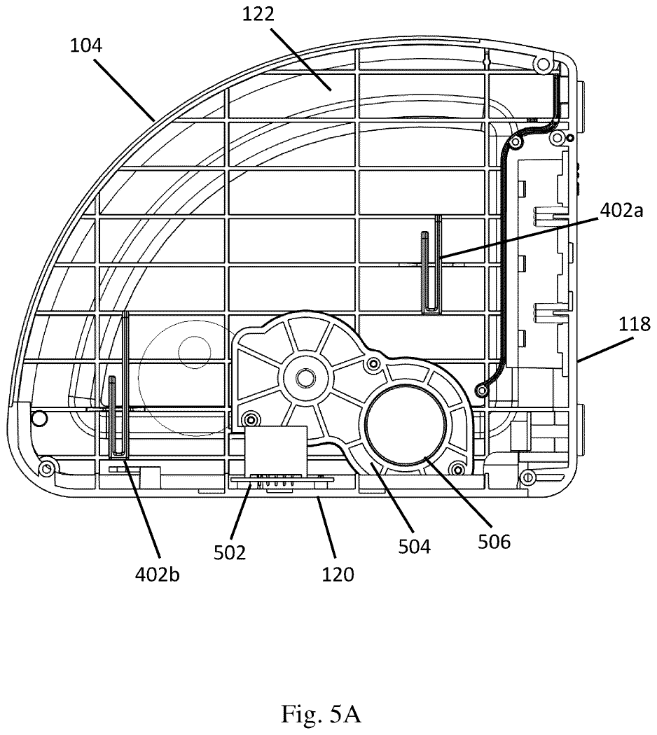

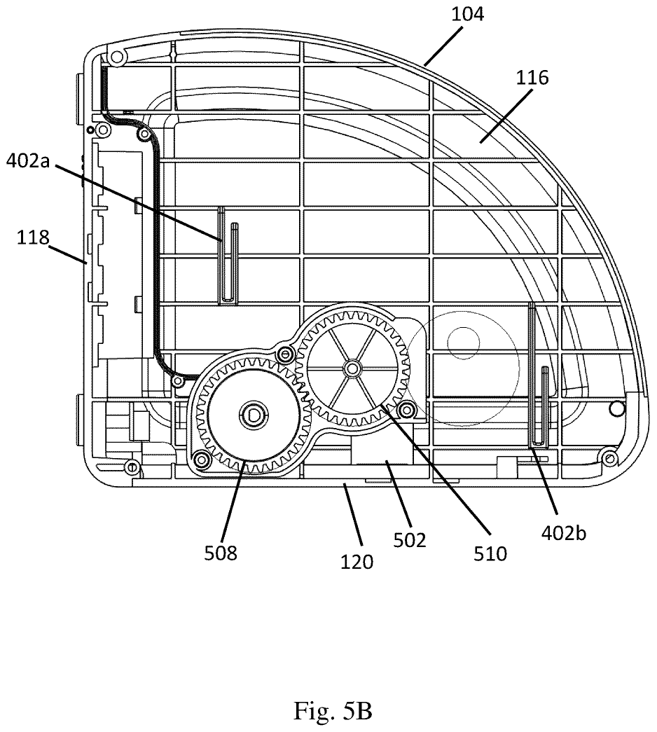

Referring now to FIGS. 5A-5B , interior side views of the housing 100, are shown with one side panel of the housing omitted for clarity to show internal components. As shown, the housing 100 can include an electrical interface 502 disposed along an interior side surface of the housing body 102 and configured to mate with the corresponding electrical interface 324 of the cartridge 300 when the cartridge is installed. The electrical interface 502 can include one or more conductive terminals, pads, or plug connectors to facilitate communication and power transfer between the housing and cartridge. The housing 100 can further include an actuator 506, such as an electric motor, operably coupled to the gear system (e.g., gear train). The actuator 506 can be configured to drive a first gear 508, which transmits torque to a second gear 510 meshed with the first gear, thereby transmitting torque to the cartridge gear 312 meshed with the second gear 510. In some embodiments, the actuator 506 is mounted to a structural frame or base within the housing. In some embodiments, the actuator 506 is supported within the housing by a cover 504 that encloses the first gear 508 and second gear 510. The cover 504 can define an opening or seat configured to receive a portion of the actuator 506 in a close or interference fit. The cover 504 can be engaged with (e.g., slidably received by) the frame 404; the cover 504 and frame 404 can have complementary contours or mating geometries, enabling the cover 504 to slide into position and lock or seat flush against the frame 404. The cover 504 can be fastened or snap-fit to the frame 404.

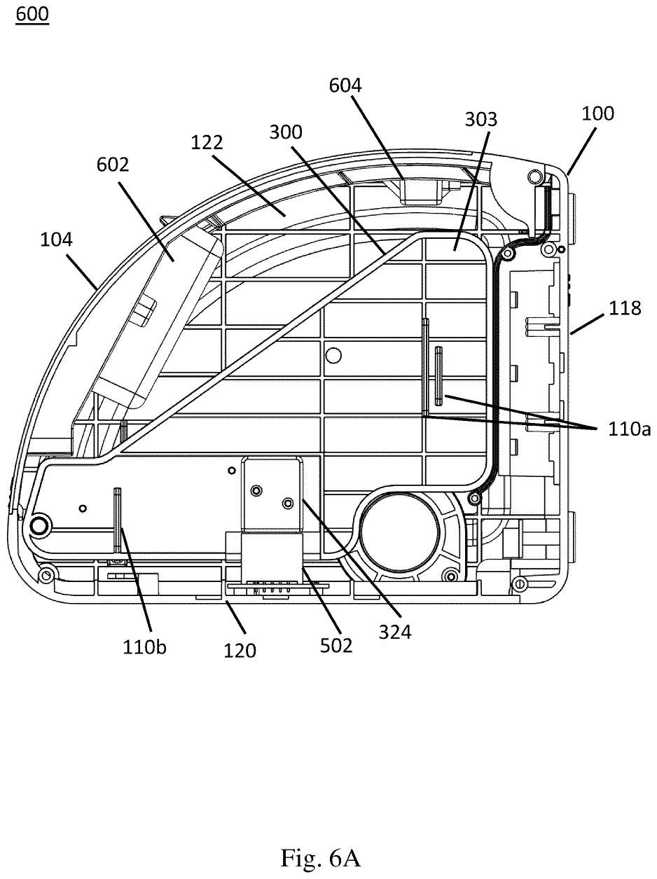

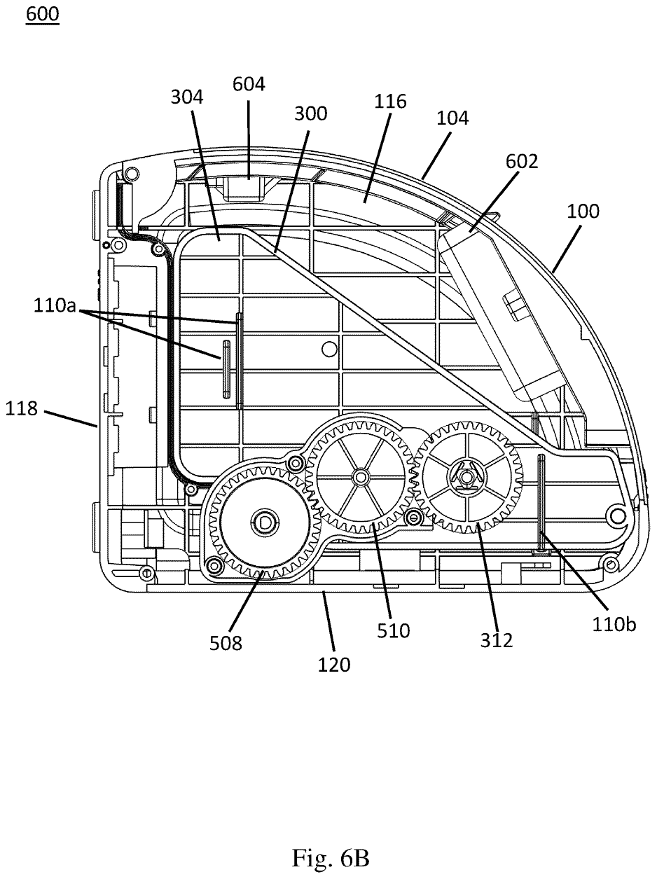

Referring now to FIGS. 6A-6B , interior side view of a system 600, including the housing 100 and the cartridge 300 are shown with one side panel omitted for clarity to show interior components. As shown, the cartridge 300 is received within the interior cavity of the housing 100 and the cartridge gear 312 is positioned to mesh with the second gear 510 of the gear train, enabling torque from the actuator 506 to be transmitted through first gear 508 and second gear 510 to the take-up spindle 313 of the cartridge 300. The electrical interface 324 of the cartridge 300 engages the electrical interface 502 of the housing 100 to establish electrical communication between the cartridge and the housing control system, allowing sensor and actuator signals to be exchanged.

In some embodiments, the housing 100 includes a sensor holder 602 disposed adjacent the path of the media and configured to support one or more sensors (e.g., sensors 106a, 106b). The sensor holder 602 can include a rectangular body or frame extending along an interior surface of the housing body 102. In some embodiments, the housing 100 includes a power control element holder 604. The sensor holder 602 and power control element holder 604 can each include a rectangular body or frame extending along an interior surface of the housing body 102. The sensor holder 602 and power control element holder 604 can each provide fixed mounting positions for the system's detection and control components.

The control system of the housing 100 can control operation of the actuator 506, the one or more sensors (e.g., sensors 106a, 106b, 320), and other electronic components of the system. The control system can receive input signals from the one or more sensors and can output control signals to the actuator 506 to regulate dispensing of the media. The control system can coordinate detection and dispensing events so that a single adhesive portion is presented for each valid user interaction while preventing unnecessary advancement of the media. The control system can determine when a sensor has detected a user's hand or stethoscope, initiate rotation of the actuator to dispense the adhesive portion, and halt actuator motion once a full adhesive portion has been exposed at the slot 108. The control system can monitor feedback from the internal sensor 320, which may detect color, opacity, or contrast indicia on the continuous sheet to identify transitions between adhesive and non-adhesive portions and thus determine when the next correct is correctly positioned.

The control system can include one or more processors, microcontrollers, or programmable logic device executing control logic stored in memory or firmware. The control system can include power-management circuity, signal-conditioning electronics, communication buses, and data interfaces electrically coupled to the actuator 506, sensors 106a, 106b, 320, and the electrical interfaces 324, 502. The control system can be implemented as a single integrated module or distributed among multiple subassemblies within the housing 100. In operation, the control system interprets sensor input and actuates the dispensing mechanism accordingly. The control system can apply predetermined timing or threshold criteria. For example, the control system can require that both sensors 106a, 106b detect an object within a specified window (e.g., between about 200 ms and 1 s) to validate a user interaction and to reduce false activations. Threshold values can be applied to sensor signal strength, reflectivity, or duration of detection to differentiate between a brief obstruction and a sustained presence corresponding to a hand or stethoscope. The control system can apply timing intervals to limit successive activations within a defined period (e.g., a lockout interval of about 1 to about 5 seconds) to prevent redundant dispensing cycles. These timing and threshold criteria can be preprogrammed or user-adjustable and ensure consistent operation across varying lighting or environmental conditions.

Referring now to FIG. 7, an illustrative view of a portion of the continuous sheet 704 of the roll of media is shown for clarity of explanation. As previously described, the continuous sheet 704 includes alternating adhesive portions and non-adhesive portions that together form a continuous strip configured for dispensing individual covers (adhesive portions). The adhesive portion 702 can correspond to a single stethoscope cover, while non-adhesive portions 705 can serve as a liner or backing region separating successive adhesive portions. FIG. 7 further illustrates that the sheet 704 can optionally include indicia 706 (e.g., a contrasting mark, printed line, or change in optical density) between adhesive and non-adhesive regions. Such indicia can be detected by sensor 320 to determine when an adhesive portion has reached the complete dispensing position. In operation, the sheet 704 can be advanced a predetermined distance based on known spacing between adjacent removable covers. Additionally or alternatively, the sensors can “read” the sheet (e.g. optically detect a line or other indica on the sheet, or the boundary of the removable cover itself) to detect when a subsequent cover has been advanced an appropriate distance such that the cover is positioned within the target zone for attachment to that stethoscope.

The system (including housing 100 and cartridge 300) can be prepared for operation by installing a cartridge 300 within the housing 100. The housing 100 can be provided. In some embodiments, the cartridge 300 is provided with a preloaded roll of media rotatably supported by the roll-supporting features 315. In some embodiments, the cartridge 300 is provided and receives a separate roll of media inserted by the user. To install the cartridge 300, a user can first open the lid 104 of the housing 100 to expose the interior cavity. The mounting projections 310a, 310b of the cartridge can be aligned with the guide slots 402a, 402b formed on the inner surfaces of the side panels 116, 122. The cartridge 300 can then be slidably inserted along the guide slots until fully seated, thereby engaging the electrical interfaces 324 and 502 and meshing the cartridge gear 312 with the second gear 510 of the gear train.

The user can manually extend and route the continuous sheet through the cartridge prior to closing the lid. A first end of the continuous sheet can be fed from the roll of media over the take-up spindle 313, through the gap between the cross-members 316 and 318, and over the cross-member 318 (such that the sheet is disposed over the sensor 320) and rod 322. The sheet can then be directed rearward toward the take-up spindle 313 and spindle shell 314, where the leading end of the sheet can be secured by insertion into a slot, clipping engagement, or frictional fit for winding. Once the sheet is properly routed, the lid 104 can be closed to position the slot 108 and protrusion 110 over the dispensing path. In the closed configuration, the slot is disposed over the cross-member 318. In some embodiments, the control system can automatically perform a brief calibration routine or advance the media slightly to ensure that a first adhesive portion is aligned with the dispensing slot 108.

The system can be used to automatically dispense a cover on a stethoscope. In use, a user can place a hand or a stethoscope head a distance (e.g., about 1 inch, about 2 inches) above the one or more sensors (e.g., sensors 106a, 106b). The one or more sensors can detect the presence of the object and/or proximity of the object. In some embodiments, the sensitivity of the sensor can be adjusted by the user to require a dwell time of detection before advancing the subsequent cover (e.g. to prevent dispensing a cover upon an object inadvertently passing through the field of view of the sensor). In response, the one or more sensors can send a corresponding signal to the control system. Upon confirming a valid detection, the control system can activate the actuator 506, which rotates the gear train and advances the continuous sheet to dispense an adhesive portion of the media. As the continuous sheet advances through the cartridge, the adhesive portion separates from the liner of the continuous sheet at a region adjacent the slot (and within the housing 100). The non-adhesive portions (e.g., liner, backing) remain within the cartridge and is directed toward the take-up spindle 313, while the adhesive portion exits the slot 108 and is supported by the protrusion 110, preventing further motion of the cover under gravity. The system can halt dispensing when the sensor 320 detects an indicium 706 or an adjacent non-adhesive portion 705, ensuring that only a single adhesive portion is dispensed for each activation.

The user can then press the stethoscope (e.g., diaphragm or bell) directly onto the exposed adhesive portion, causing the cover to adhere securely to the stethoscope surface. The user can then withdraw the stethoscope, separating the adhesive portion from the liner of the continuous sheet and leaving the cover applied to the stethoscope. In some embodiments, the system can include an indicator (e.g., visible or audible indicator) to confirm successful dispensing. In some embodiments, the system can include an indicator to alert the user when the cartridge is empty (i.e., when the roll of media is spent and there are no more covers available for dispensing).

In some embodiments, the system dispenses adhesive portions (covers) through a manual or user-actuated input instead of, or in addition to, using sensors. For example, a button or switch disposed on the housing can be configured to trigger the actuator 506 when pressed by activated (e.g., pressed) by a user. Activation of the button or switch can generate a control signal that causes the actuator to rotate the first gear and cause advancement of a single adhesive portion in a similar manner as sensor-based activation.

While the systems and methods disclosed herein have been primarily described in the context of dispensing a cover for a stethoscope diaphragm it will be appreciated that the same principles can be applied to other components of a stethoscope or to other medical or non-medical instruments. For example, in some embodiments, the system can be configured to dispense a cover for a stethoscope bell. In another example, the system can be configured for dispensing covers for ultrasound transducers, or other surfaces requiring temporary coverage between uses.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A system for dispensing a cover comprising an adhesive portion for application to a stethoscope, comprising:
a cartridge comprising:
a body having an exterior surface and an interior surface;
a roll of media rotatably coupled to the body, the roll of media comprising a continuous sheet having removable adhesive portions and non-adhesive portions; and
a first gear operably coupled to the roll of media, such that a rotation of the first gear rotates the roll of media;
a housing, the housing having a body with an exterior surface and an interior surface, the body configured to receive the cartridge;
one or more sensors coupled to the housing and configured to detect a presence of an object adjacent the housing; and
a second gear operably coupled to an actuator disposed within the housing, the second gear configured to mesh with the first gear, wherein a rotation of the second gear rotates the first gear,
wherein detection of the presence of the object by the one or more sensors causes dispensing of an adhesive portion of the continuous sheet through a slot of the housing.

2. The system of claim 1, the housing further comprising a lid pivotably coupled to the body of the housing.

3. The system of claim 2, wherein the lid comprises a curved surface.

4. The system of claim 3, wherein the slot is disposed tangentially relative to a curvature of the lid, such that an adhesive portion of the continuous sheet is dispensed along the curved surface of the lid.

5. The system of claim 2, wherein the one or more sensors are disposed on the lid.

6. The system of claim 2, further comprising a protrusion disposed adjacent the slot, the one or more sensors disposed between the slot and the protrusion.

7. The system of claim 1, further comprising a third gear operably coupled between the actuator and the second gear.

8. The system of claim 7, wherein the actuator is directly coupled to the third gear such that rotation of an output shaft of the actuator directly rotates the third gear.

9. The system of claim 7, wherein the third gear is configured to mesh with the second gear.

10. The system of claim 1, wherein the adhesive portion comprises a circular region and a linear region extending from the circular region.

11. The system of claim 1, further comprising a machine-readable code, the machine-readable code disposed on the exterior surface of the housing.

12. The system of claim 1, wherein the cartridge further comprises a plurality of mounting projections extending outwardly from the exterior surface of the body of the cartridge.

13. The system of claim 12, wherein the housing further comprises a plurality of guide slots formed on the interior surface of the body of the housing, each one of the plurality of guide slots configured to receive one of the plurality of mounting projections of the cartridge.

14. The system of claim 1, further comprising one or more sensors coupled to the cartridge, the one or more sensors configured to detect a predetermined opacity of the continuous sheet.

15. The system of claim 1, further comprising a first electrical interface disposed within the housing and a second electrical interface disposed on the cartridge, the first electrical interface and the second electrical interface configured to engage and electrically communicate when the cartridge is received within the housing.

16. A cartridge for use in a system for dispensing a cover comprising a removable adhesive portion for application to a stethoscope, comprising:
a body having an exterior surface and an interior surface;
a roll of media rotatably coupled to the body, the roll of media comprising a continuous sheet having removable adhesive portions and non-adhesive portions; and
a first gear operably coupled to the roll of media, such that a rotation of the first gear rotates the roll of media.

17. The cartridge of claim 16, wherein the body comprises a pair of opposing side panels and a plurality of cross-members extending between the side panels.

18. The cartridge of claim 16, further comprising a take-up spindle disposed within the body and operably coupled to the first gear.

19. The cartridge of claim 18, further comprising a spindle shell disposed over the take-up spindle.

20. The cartridge of claim 16, wherein a first end of the continuous sheet is coupled to the spindle shell.
